(19) United States
(12) Patent Application Publication
(10) Pub. No.: US 2003/0219807 A1
(43) Pub. Date: Nov. 27, 2003

(54) Multiple genes relevant for the characterization, diagnosis, and manipulation of stroke

(76) Inventors: Hermann Lubbert, Leverkusen (DE); Peter Engels, Bergisch Gladbach (DE); Stefan Zwilling, Leverkusen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/392,808

(22) Filed: Mar. 19, 2003

Related U.S. Application Data
(60) Provisional application No. 60/366,353, filed on Mar. 20, 2002.

(51) Int. Cl. C12Q 1/68

(52) U.S. Cl. 435/6

(57) ABSTRACT

The present invention relates to the use of differentially expressed polynucleotide sequences or polypeptides for the characterization of stroke or progression thereof or progression of neurodegenerative processes as consequence thereof, a method for characterizing stroke, a method for identifying therapeutic agents for stroke and the use of such sequences for the development of a medicament.

### RELATED APPLICATION

This application claims priority under 35 U.S.C. §119(e) to U.S. Provisional Application No. 60/366,353, filed Mar. 20, 2002.

### FIELD OF THE INVENTION

The present invention relates to the use of differentially expressed polynucleotide sequences or polypeptides for the characterization of stroke or progression thereof or progression of neurodegenerative processes as consequence thereof, a method for characterizing stroke, a method for identifying therapeutic agents for stroke and the use of such sequences for the development of a medicament.

### BACKGROUND OF THE INVENTION

Stroke is a rather common and potentially harmful event that often leaves patients severely disabled for the rest of their lives. It is the result of either an interruption of blood and therefore oxygen supply to the brain, or bleeding in the brain that occurs when a blood vessel bursts. The reduced oxygen supply leads to shortage of energy in the affected brain regions and results in the death of neurons around the lesion. The degeneration spreads from the site that is affected by the reduced blood supply into regions which have at all times obtained sufficient oxygen.

Stroke results from a loss of blood flow to the brain caused by thrombosis or haemorrhage. With an incidence of 250-400 in 100.000 and a mortality rate of around 30%, stroke is a major public health problem. About one-half of the stroke survivors suffer from significant persisting neurological impairment and/or physical disability. Thus, the economic costs of stroke amount to many billions of dollars worldwide.

A stroke occurs when the blood supply to part of the brain is suddenly interrupted or when a blood vessel in the brain bursts. As a consequence, brain cells die when they no longer receive oxygen and nutrients from the blood or when they are damaged by sudden bleeding into the brain. Some brain cells die immediately after interruption of the blood flow into the brain, while others remain at risk for death and stay in a compromised state for hours. These damaged cells make up the so-called “ischemic penumbra”, and with timely treatment these cells could be saved. Stroke ultimately leads to infarction, the death of huge numbers of brain cells, which are eventually replaced by a fluid-filled cavity (or infarct) in the injured brain.

There are two major forms of stroke: ischemic—blockage of a blood vessel supplying the brain, and hemorrhagic—bleeding into or around the brain. An ischemic stroke can be caused by a blood clot (embolus or thrombus), which is blocking a vessel. It can also be caused by the narrowing of an artery due to the build-up of plaque (a mixture of fatty substances, including cholesterol and other lipids). The underlying pathological process called stenosis is often observed in arteriosclerosis, the most common blood vessel disease. About 80% of all strokes are ischemic strokes. A hemorrhagic stroke is caused by the bursting of an artery in the brain. Subsequently, blood spews out into the surrounding tissue and upsets not only the blood supply but also the delicate chemical balance neurons require to function. Hemorrhagic strokes account for approximately 20% of all strokes.

A transient ischemic attack (TIA), sometimes called a mini-stroke, starts just like a stroke but then resolves leaving no noticeable symptoms or deficits. The occurrence of a TIA is a warning that the person is at risk for a more serious and debilitating stroke. About one-third of patients who have a TIA will have an acute stroke sometime in the future. The addition of other risk factors compounds a person's risk for a recurrent stroke. The average duration of a TIA is a few minutes. For almost all TIAs, the symptoms go away within an hour. There is no possibility to distinguish whether symptoms will be just a TIA or persist and lead to death or disability.

Recurrent stroke is frequent; about 25 percent of people who recover from their first stroke will have another stroke within 5 years. Recurrent stroke is a major contributor to stroke disability and death, with the risk of severe disability or death from stroke increasing with each stroke recurrence. The risk of a recurrent stroke is greatest right after a stroke, with the risk decreasing with time. About 3 percent of stroke patients will have another stroke within 30 days of their first stroke and one-third of recurrent strokes take place within 2 years of the first stroke.

The most important risk factors for stroke are hypertension, arteriosclerosis, heart disease, diabetes, and cigarette smoking. Others include heavy alcohol consumption, high blood cholesterol levels, illicit drug use, and genetic or congenital conditions, particularly vascular abnormalities. People with multiple risk factors compound the destructive effects of these risk factors and create an overall risk greater than the simple cumulative effect of the individual risk factors.

Although stroke is a disease of the brain, it can affect the entire body. Depending on the affected brain region and the severity of the attack, post-stroke patients suffer from a variety of different symptoms. Some of the disabilities that can result from stroke include paralysis, cognitive deficits, speech problems, emotional difficulties, daily living problems, and pain. Stroke disability is devastating to the stroke patient and family, but therapies are available to help rehabilitate post-stroke patients. The mortality rate observed in ischemic stroke is around 30%. The time window for a medical treatment is narrow and limited to anticoagulants and thrombolytic agents, which must be given immediately (at latest 3 hours) after having a stroke. Unfortunately, there is no effective neuroprotective medication available, which is able to stop the delayed degeneration of neurons following the initial stroke attack.

Stroke symptoms appear suddenly. The following acute symptoms can be observed. Sudden numbness of the face, arm or leg, difficulties in talking or understanding speech, trouble seeing in one or both eyes, sudden trouble in walking, loss of balance and coordination. Severe headache with no known cause does also occur. Even more importantly, there is a variety of severe disabilities occurring and persisting in post-stroke patients. Paralysis is a frequent disability resulting from stroke. Cognitive deficits (problems with thinking, awareness, attention and learning) are also commonly observed. Post-stroke patients exhibit language deficits and also emotional deficits (like post-stroke depression). Furthermore, an uncommon type of pain, called central pain syndrome (CPS), can occur after having a stroke.

Currently the only effective treatment for thrombotic stroke is the use of anticoagulants (e.g. heparin), and thromobolytics (recombinant tissue plasminogen activator). Neuroprotective agents, which are effective in animal models, have generally proved ineffective in the clinic, and none are yet registered for use in stroke.

A number of experimental models have been developed for global ischemia and focal ischemia. The availability of these different models provides an opportunity to investigate mechanisms of stroke. Finding common features in different models or over several time points within one model should pro-vide better insight into the mechanisms critical for stroke, and comparison of the models should help to understand development, progression and consequences of stroke.

Most common global ischemia models are:

a) The Two Vessel Model

Models of transient global ischemia resulting in patterns of selective neuronal vulnerability are models that attempt to mimic the pathophysiology of cardiac arrest or hemodynamic conditions that result from severe systemic hypotension. Reversible high-grade forebrain ischemia is generated by bilateral common carotid artery (CCA) occlusion. Together with systemic hypo-tension conditions it reduces the blood flow to severe ischemic levels (Smith et al. 1984 Acta Neuropathol 64:319-332). This model of transient global ischemia has the advantage of a one stage surgical preparation, the production of a high-grade forebrain ischemia and the possibility to conduct chronic survival studies in order to assess the potential of neuroprotective drugs.

b) The Four Vessel Model

This model results in a high-grade forebrain ischemia but is produced in two stages, one to manipulate each of the CCA, and the second stage 24 h later to produce the forebrain ischemia. The advantage is that the second step can be produced in awake freely moving animals (Pulsinelli et al. 1982 Ann Neurol 11:491-498). Similar pathohistological results could be obtained for both vessel models.

c) The Cardiac Arrest Model

Forebrain ischemia models are of value to study cerebral ischemia but these models do not exactly mimic the hemodynamic consequences of a cardiac arrest, which results in a complete ischemia of the brain, spinal cord and extracerebral organs (Katz et al. 1995 J Cereb Blood Flow Metab 15:1032-1039). The initial cardiac arrest models from Safar et al. (1982 Protection of tissue against hypoxia Elsevier Biomedical Press; 147-170) or Korpaczew et al. (1982 Partol Fizjol Eksp Ter 3:78-80), were developed further by Katz et al. (1989 Resuscitation 17:39-53) and Pluta et al. (1991 Acta Neuropathologica 83:1-11) to models with controllable insult. Katz and colleagues (1995) have reported a reproducible outcome model of cardiac arrest with apneic asphyxia of 8 min, leading to the cessation of circulation at 3-4 min of apnea and resulting in cardiac arrest of 4-5 min. At 72 hr after injury, widespread patterns of ischemic neurons were found in many brain regions, including cerebral cortex, caudate putamen CA1 and CA3 regions of hippocampus, thalamus, cerebellum and brain stem.

Pluta et al. described a primary mechanical cardiac arrest model whereby global ischemia was induced by cardiac arrest for 3 to 10 min with survival periods of the animals from 3 min to 7 days.

Although these models have several limitations, they provide a method for studying the mechanisms of neuronal injury resulting from the clinically realistic cerebral insult and screening potential cerebral resuscitation therapies.

Focal Ischemia Models

Models of permanent or transient focal ischemia typically giving rise to localized brain infarction have routinely been used to investigate the pathophysiology of stroke. For example, models of middle cerebral artery (MCA) occlusion in a variety of species have gained increased acceptance due to their relevance to the human clinical setting.

a) the Permanent MCA Occlusion Models

Tamura and colleagues (1981 J Cereb Blood Flow Metab 1:53-60) developed a subtemporal approach as standard model of proximal MCA occlusion. In models of permanent MCA occlusion, electrocauterization of the MCA proximal to the origin of the lateral lenticulostriate arteries is utilized routinely. In these models, severe reductions in blood flow are seen within the ischemic core, with milder reductions in blood flow within the border or penumbral regions. The addition of moderate arterial hypotension has the effect of enlarging infarct volume.

b) The Transient MCA Occlusion Models

In human ischemic stroke, recirculation frequently occurs after focal ischemia. Thus, models of transient MCA occlusions have also been developed, mainly in rats or mice, whereby surgical clip or sutures are introduced to induce a transient ischemic insult.

Some further animal models for stroke are considered in several reviews like the articles of W. D. Dietrich (1998 Int Review of Neurobiology 42:55-101), Wiebers et al. (1990 Stroke 21:1-3) or Zivin and Grotta (1990 Stroke 21:981-983).

### SUMMARY OF THE INVENTION

Object of the present invention is to identify and characterize development, conditions (status which elicits), progression and consequences of stroke on a molecular basis.

This object is met by the use of polynucleotide sequences selected from the group of sequences SEQ ID NO: 1 to 88 or homologues or fragments thereof or the according polypeptides for the characterization of a) development and/or occurrence of stroke, b) the progression of the pathology of stroke and/or b) the consequences of the pathology of stroke, whereby the characterization is carried out outside of a living body.

Polynucleotide sequences SEQ ID NO: 1 to 88 are expressed sequence tags (ESTs) representing genes, which are differentially expressed under stroke, particularly under global ischemia in the cardiac arrest model (Pluta et al. 1991 Acta Neuropathol 83:1-11).

The model is explained in more detail in the literature and in the examples below.

### BRIEF DESCRIPTION OF THE DRAWINGS

FIG. 1 shows changes in expression of SEQ ID NO: 37.

FIG. 2 shows changes in expression of SEQ ID NO: 79.

FIG. 3 shows changes in expression of SEQ ID NO: 35.

FIG. 4 shows changes in expression of SEQ ID NO: 57.

FIG. 5 shows changes in expression of SEQ ID NO: 70.

FIG. 6 shows changes in expression of SEQ ID NO: 66.

FIG. 7 shows changes in expression of SEQ ID NO: 3.

### DETAILED DESCRIPTION OF THE PREFERRED EMBODIMENT

The term “polynucleotide sequence” or “nucleic acid sequence” designates in the present application any DNA or RNA sequence, independent of the length. Thus this term can describe short sequences like PCR primers or probes for hybridization, as well as whole genes or cDNA of these genes.

The term “polypeptide” or “amino acid sequence” designates a chain of amino acids, independent of their length, however, in any case more than one amino acid.

As “homologues” of polynucleotide sequences such polynucleotide sequences are designated which encode the same type of protein as one of the polynucleotide sequences described herein. Accordingly as “homologues” of a polypeptide the polypeptides are designated, which have an amino acid sequence, wherein at least 70%, preferably 80%, more preferably 90% of the amino acids are identical to one of the proteins of the present invention and wherein the replaced amino acids preferably are replaced by homologous amino acids. As “homologous” amino acids are designated those, which have similar features concerning hydrophobicity, charge, steric features etc. Most preferred are amino acid sequences, containing the species- or family-dependent differences of the amino acid sequence. Particularly as “homologues” sequences are designated those, which correspond to one of the cited sequences in another species or individual. For example if in the present invention a rat model is used and the cited polynucleotide sequence encodes the rat protein, the according polynucleotide sequence and protein of a mouse or of a human is designated as “homologue”. Further splice variants and members of gene families are designated as homologues.

“Fragments” of a polynucleotide sequence are all polynucleotide sequences, which have at least 10 identical base pairs compared to one of the polynucleotide sequences shown in the present application or by the genes represented by these polynucleotide sequences. The term “fragment” encloses therefore such fragments as primers for PCR, probes for hybridization, DNA fragments included in DNA vectors like plasmids, cosmids, BACs or viral constructs, as well as shortened splice variants of the genes identified herein. As a fragment of a protein (polypeptide) amino acid sequences are designated which have at least three amino acids, preferably at least 10 amino acids. Therefore fragments serving as antigens or epitopes are enclosed in this designation.

In the present application the term “sequence” is used when either a polynucleotide sequence (=nucleic acid sequence) or a polypeptide (=amino acid sequence) or a protein is meant. That means, when it is irrelevant which type of sequence is used the type is not designated particularly, but with the more common term “sequence”.

In the present application the term “stroke” means the development, occurrence, progression and consequences of the disease state. Several features of the development, occurrence and consequences of this disease are described herein above.

The basis of the models and methods described in the present application is the examination and determination of the expression of genes, which are differentially expressed during development, conditions, progression and consequences of stroke. Therefore for the examination each sequence can be used which allows the determination of the expression rate of the considered gene. Such a sequence can be at least one of the polynucleotide sequences SEQ ID NO: 1 to 88 or homologues or fragments thereof, as well as the polypeptides encoded thereby, however, just as well polynucleotide sequences and the according polypeptides can be used which are (parts of) the genes represented by the polynucleotide sequences SEQ ID NO: 1 to 88.

According to the invention it has been found, that the genes represented by the polynucleotide sequences SEQ ID NO: 1 to 88 are differentially expressed in the cardiac arrest model of stroke.

Therefore the present invention provides sequences, which represent genes, which are differentially expressed under stroke. Such polynucleotide sequences and the according polypeptides allow the determination and examination of stroke. Most of these sequences have not yet been regarded in relation to stroke. Sequences which are known to be differentially expressed in connection with stroke conditions are Apolipoprotein E, herein referred to as SEQ ID NO: 40, (2001 J Cereb Blood Flow Metab 21:1199-1207), β-amyloid precursor protein (APP), herein referred to as SEQ ID NO: 77 (1996 Neuroreport 7:2727-2731), Preproenkephalin, herein referred to as SEQ ID NO: 35 (1997 Brain Res 744:185-187), Cathepsin B, herein referred to as SEQ ID NO: 86 (1997 J Neurosurg 87:716-723).

For these examinations animal models can be used. As such a model any animal can be used wherein the necessary preparations can be carried out, however mammalian models are preferred, even more preferred are rodents. Most preferred animal models of the present invention are rat and mouse models.

The sequences of the present invention further can be used for diagnosing stroke of a human outside of the living body by determining the expression levels of at least one of the cited sequences in comparison to the non-disease status. During treatment period of a patient the expression of the presently shown sequences can also be used outside of the body for assessing the efficacy of stroke treatment. In this case blood, cerebrospinal fluid (CSF) or tissue is removed from the patient and expression is determined in the samples.

For determination and comparison of the expression levels of at least one of the genes identified in the present invention any of the commonly known methods can be used, either on RNA/cDNA level or on protein level. For example PCR, hybridization, micro array based methods, western blot or 2-D protein gel analysis are suitable methods. One preferred method is the digital expression pattern display method (DEPD method), explained in detail in WO99/42610. The method used for determination of expression levels is not restrictive, as long as expressed amounts can be quantified.

The sequences of the present invention can further be used to develop new animal models for stroke. By examination of the expression levels of at least one of the shown sequences, a procedure might be determined, which is useful for generating a suitable animal model for different interesting conditions. In particular, useful animal models might be transgenic, knock out, or knock in models.

In such a newly generated animal model as well as in one of the known models the efficacy of compounds can be tested, using techniques known in the art. As well assay systems can be used that are based on the shown sequences. Such assay systems may be in vivo, ex vivo or in vitro assays. In any case the models or assay systems are contacted with the compound(s) to be tested and samples are obtained from these models/systems, wherein expression levels of the sequences are determined and compared to the non-treated model/system.

Dependent of the model used the samples can be derived from whole blood, CSF or whole tissue, from cell populations isolated from tissue or blood or from single cell populations (i.e. cell lines).

In one embodiment of the invention cellular assays can be used. Preferred cells for cellular assays are eukaryotic cells, more preferably mammalian cells. Most preferred are neuronal-like cells, like SHSY5Y (neuroblastoma cell line), hippocampal murine HT-22 cells, primary cultures from astrocytes, cerebral cortical neuronal-astrocytic co-cultures, mixed neuronal/glial hippocampal cultures, cerebellar granular neuronal cell cultures, primary neuronal cultures derived from rat cortex (E15-17), or COS cells (African green monkey, kidney cells); CHO cells (Chinese hamster ovary), or HEK-293 cells (human embryonic kidney).

Whereas the comparison of the expression levels (disease/non-disease status) of at least one of the provided sequences might give information about the examined disease status, it is preferred to determine the expression levels of more than one of the sequences simultaneously. Thus several combinations of the sequences can be used at different time points. By combination of several sequences a specific expression pattern can be determined indicating and/or identifying the conditions of the disease. The more expression rates are determined simultaneously, the more specific the result of the examination might be. However, good results also can be obtained by combination of only a few sequences. Therefore for the present invention it is preferred to compare the expression rates of at least two of the sequences provided herein, more preferred of at least 4, further more preferred of at least 6 of the sequences.

Since the presently provided sequences represent genes, which are differentially expressed, the expression rates of the single genes can be increased or decreased independently from each other. “Independently” in this context means that the expression rate of each of the genes can but need not be influenced by each other. In any case expression levels different from the non-disease status might be a hint to the disease status, which is examined.

The disease status, which is considered in the present invention, is stroke. The preferred types of stroke are ischemic and hemorrhagic stroke. Consequences, which might be related to stroke, are among others severe headache, paralysis, cognitive deficits, speech problems, emotional difficulties, daily living problems, and central pain syndrome.

Independent whether stroke is diagnosed or characterized, a model for stroke is characterized, the efficacy of stroke treatment or the efficiency of a compound in a model shall be examined, the determination of the expression levels of at least one of the sequences is carried out outside of a living body. A method to obtain such results comprises: providing a sample comprising cells or body fluids expressing one or more genes represented by polynucleotide sequences selected from the group of SEQ ID NO: 1 to 88 or homologues or fragments thereof; detecting expression of one or more of the genes in said cells; comparing the expression of the genes in the test cells to the expression of the same genes in reference cells whose expression stage is known; and identifying a difference in expression levels of the considered sequences, if present, in the test cell population and the reference cell population.

As mentioned above, detection of the expression of the genes can be carried out by any method known in the art. The method of detection is not limiting the invention.

Expression levels can be detected either on basis of the polynucleotide sequences or by detecting the according polypeptide, encoded by said polynucleotide sequence.

Preferred methods for detection and determination of the gene expression levels are PCR of cDNA, generated by reverse transcription of expressed mRNA, hybridization of polynucleotides (Northern, Southern Blot systems, In situ hybridization), DNA-microarray based technologies, detection of the according peptides or proteins via, e.g., Western Blot systems, 2-dimensional gel analysis, protein micro-array based technologies or quantitative assays like e.g. ELISA tests.

The most preferred method for quantitative analysis of the expression levels is the digital expression pattern display method (DEPD), described in detail in WO99/42610.

The sequences of the present invention can further be used for identifying therapeutic agents and their efficacy for treating stroke. For example a method can be used comprising: providing a test cell population comprising cells capable of expressing one or more genes represented by nucleic acid sequences selected from the group consisting of SEQ ID NO: 1 to 88 or homologues or fragments thereof; contacting said test cell population with the test therapeutic agent; detecting the expression of one or more of the genes in said test cell population; comparing the expression of the gene(s) in the test cell population to the expression of the gene(s) in a reference cell population whose disease stage is known; and identifying a difference in expression levels of the considered sequences, if present, in the test cell population and the reference cell population, thereby identifying a therapeutic agent for treating stroke.

Test cells can be obtained from a subject, an animal model or cell cultures of fresh cells or cell lines. Further in vitro assays may be used.

A method examining the different expression patterns of the differentially expressed gene(s) therefore can be used for testing agents and compounds for their efficiency for treatment of stroke. Which model is used is not relevant, as long as the model allows the determination of differences in expression amounts.

In such a model cells are contacted with the interesting agent or compound and expression of at least one of the genes considered in the present invention is determined in comparison to the expression of the same gene in cells which never have been contacted to the according agent/compound. Contacting the cells either can be affected by administering the agent/compound to an animal or by contacting isolated cells of tissue, CSF, or blood or cells of cell lines in culture with the agent/compound.

By examination of the influence the considered agent(s)/compound(s) have on the expression of at least one of the genes, the efficacy of the agent(s)/compound(s) can be estimated. This allows the decision whether it is worthwhile to develop a medicament containing such an agent or compound.

Whether the expression is determined on basis of mRNA generation or on basis of protein generation is not relevant, as long as the difference of the expression rate can be determined. Therefore both, the polynucleotide sequences, and the polypeptides or proteins shown in the present application can be used for the development or the identification of a medicament.

The development of a medicament can be desirable for example if the considered compound/agent has any influence on the regulation of the expression rate or on the activity of any polynucleotide sequence or polypeptide/protein of the present invention. Said influence can be for example acceleration, promotion, increase, decrease or inhibition of the expression or activity.

Said influence of a compound or agent can be examined by a method comprising contacting a sample comprising one of the nucleic acid sequences or of the polypeptides of the present invention with a compound that binds to said sequence in an amount sufficient to determine whether said compound modulates the activity of the polynucleotide or polypeptide/protein sequence.

By such a method a compound or agent modulating the activity of any of the nucleic acid sequences or any polypeptides of the present invention can be determined.

Furthermore the sequences itself can be used as a medicament.

An example for such a use is the use of a polynucleotide sequence as an antisense agent. Antisense agents, including but not limited to ribonucleotide or desoxyribonucleotide oligomers, or base-modified oligomers like phosphothioates, methylated nucleotides, or PNAs (peptide nucleic acids), can hybridize to DNA or mRNA, inhibiting or decreasing transcription or translation, respectively. Thus, polynucleotide sequences of a gene, which is increased in expression rate under stroke, can be used as antisense agents to decrease the expression rates of said gene. Further such polynucleotide sequences can be used for gene therapy.

Another example for such a use is the use of a polypeptide or a protein as a medicament. In case that the expression of a gene is decreased under stroke and therefore not “enough” protein is provided by the body to maintain natural (healthy) conditions, said protein can be administered as a medicament. In case a gene is increased under stroke, representing a protective beneficial or adaptive response of the brain, this effect can be further strengthened by adding even more of the corresponding protein as medicament.

A pharmaceutical composition comprising a polynucleotide sequence or a polypeptide according to the present invention can be any composition, which can serve as a pharmaceutical one. Salts or aids for stabilizing the sequences in the composition preferably are present.

For the determination of the expression of the relevant genes the generated sequences have to be detected. Therefore several reagents can be used, which are for example specific radioactive or non-radioactive (e.g., biotinylated or fluorescent) probes to detect nucleic acid sequences by hybridization, e.g., on DNA microarrays, primer sets for the detection of one or several of the nucleic acid sequences by PCR, antibodies against one of the polypeptides, or epitopes, or antibody- or protein-microarrays. Such reagents can be combined in a kit, which can be sold for carrying out any of the described methods.

Further the sequences defined in the present invention can be used to “design” new transgenic animals as model for stroke. Therefore the animals are “created” by manipulating the genes considered in the present application in a way that their expression in the transgenic animal differs from the expression of the same gene in the wild-type animal. In which direction the gene expression has to be manipulated (up- or down-regulation) depends on the gene expression shown in the present application. Methods of gene manipulation and methods for the preparation of transgenic animals are commonly known to those skilled in the art.

For further examinations or experiments it might be desirable to include any of the nucleic acids of the present invention into a vector or a host cell. By including the sequences in a host cell for example cellular assays can be developed, wherein the genes, polynucleotide sequences and the according proteins/polypeptides further can be used or examined. Such vectors, host cells and cellular assays therefore shall be considered as to fall under the scope of the present invention.

The following examples are provided for illustration and are not intended to limit the invention to the specific example provided.

### EXAMPLE 1

### Preparation of Rat Cardiac Arrest Model

Cardiac arrest was performed in female Lewis-rats at 3 months of age (150-220 g), resulting in total cessation of blood flow leading to global cerebral ischemia. After 10 min of ischemia, the animals were resuscitated by external heart massage and ventilation. The group size was 2×3 animals.

For induction of cardiac arrest, a special blunt-end, hook-like probing device was inserted into the right parasternal line across the third intracostal spaces into the chest cavity. Next, the probe was gently pushed down the vertebral column until a slight resistance from the presence of the right pulmonary veins was detected. The probe then was tilted 10-20° caudally. Then, the probe was rotated in a counter-clockwise direction about 135-140° under the inferior vena cava. At this position, the occluding part of the device was positioned right under the heart vessel bundle (inferior vena cava, superior right and left vena cava, ascending aorta, and pulmonary trunk). The pulmonary veins (left and right) were closed by the rotation of the occluding part of the hook. In the last step, the probe was pulled up with concomitant compression of the heart vessel bundle against the sternum. The end of the occluding portion of the hook was then positioned in the left parasternal line in the second intercostal space. To prevent upward movement of the chest and to insure complete ligation of the vessels, simple finger pressure was applied downward the sternum, producing total hemostasis and subsequent ventricular arrest. The effect of the whole procedure is total cessation of both arterial and venous blood flow, it essentially represents the onset of clinical death. After 2.5-3.5 min, the probe was released and removed from the chest by reverse procedural succession, and the animals remained in this position until the beginning of resuscitation.

The resuscitation procedure consisted of external heart massage until spontaneous heart function was recovered and controlled respiration occurred. During this time, air was pumped through a polyethylene tube inserted intratracheally that was connected to a respirator. External heart massage was produced by the index and middle fingers rapidly striking against the chest (sternum) for 1-2 min at the level of the fourth intracostal area with a frequency of 150-240/min in continuous succession. The ratio of strikes to frequency of ventilation was 6:1 or 8:1.

An electrocardiogram (lead II-EEG) was recorded continuously during the course of the experiment. Moreover, heart activity was monitored using a loud speaker connected to the output lead of the electroencephalograph. Additionally, the cranial bones were exposed at the sagittal and coronal sutures, where silver-needle electrodes were attached for recording on an electrocorticogram (ECoG). All measurements were registered on a ten-channel electroencephalogram (Accutrace-100A, Beckmann).

2×3 sham operated animals served as controls. These animals were treated similarly to the experimental group with one major exception. Under anesthesia, the probe was inserted through the chest wall into the plural cavity as has already been described above but without further manipulation and torsion of the probe. The probe remained in the chest for essentially the same time period (3.5 min) as in the experimental group. The control animals were then returned to their cages for recovery.

Tissue preparation occurred 0.5 hr, 1 hr, 6 hrs, 3 days, 7 days and 2 years after surgery. Tissues were frozen on liquid nitrogen prior to RNA preparation.

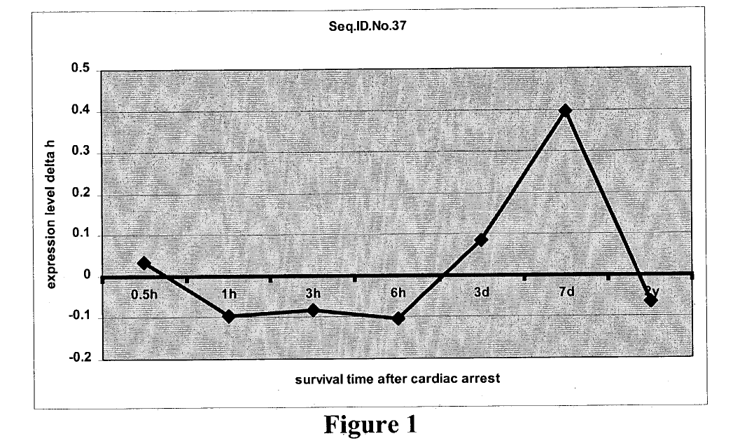

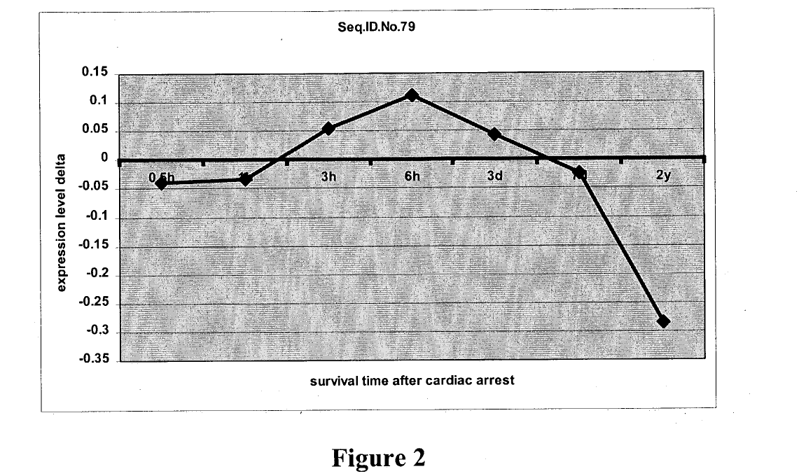

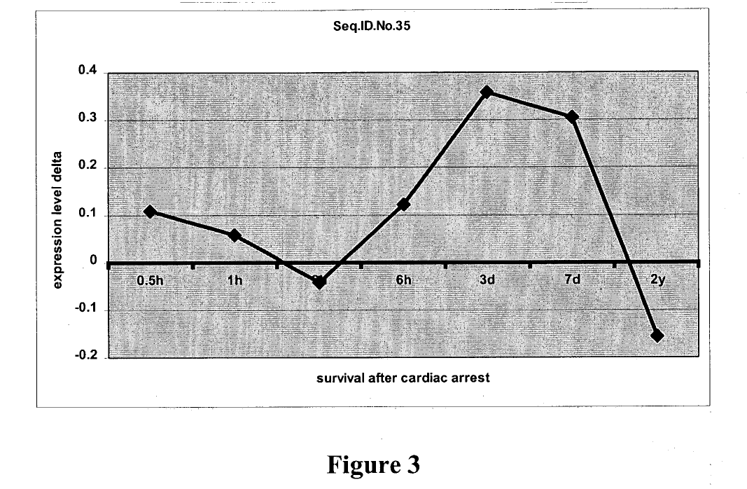

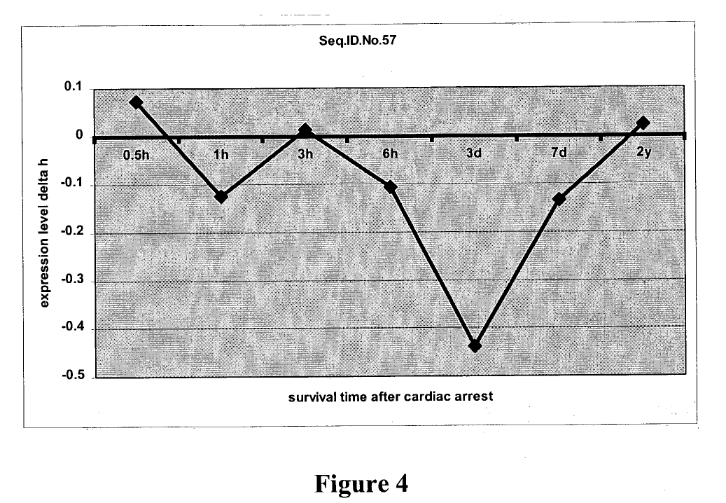

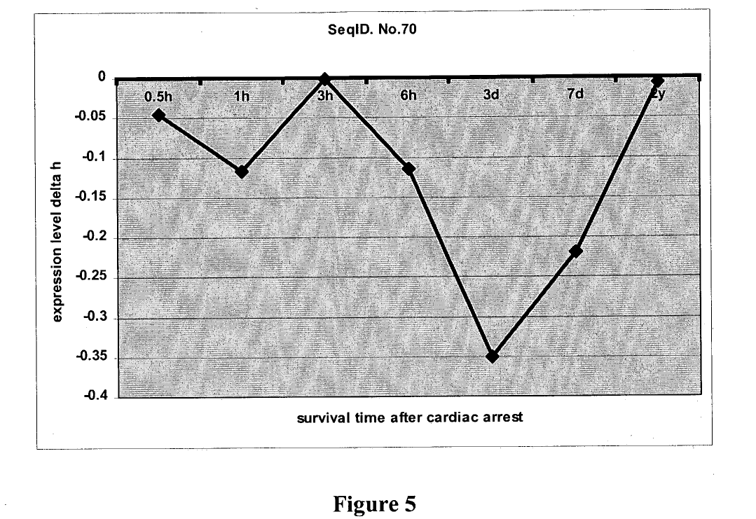

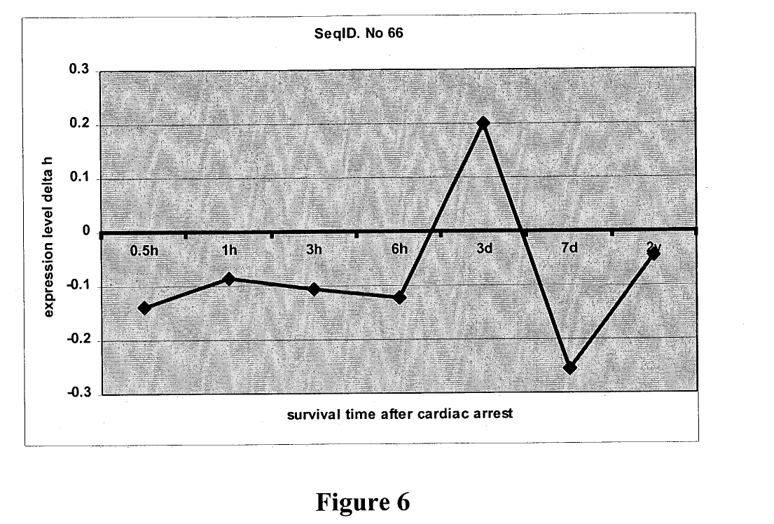

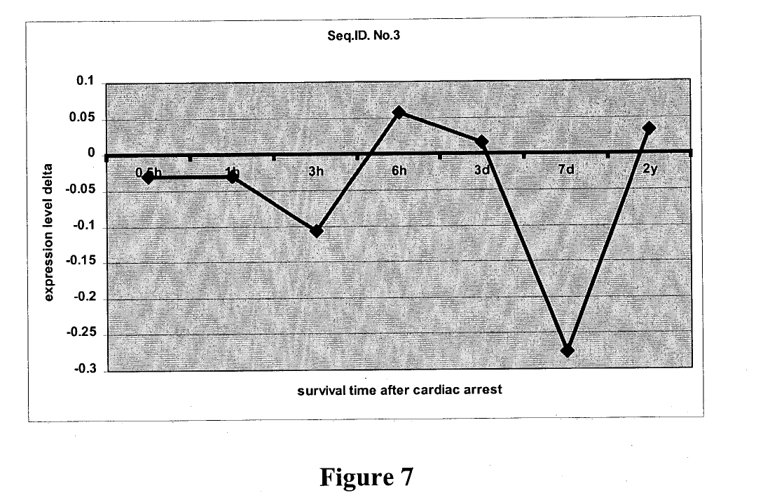

FIGS. 1 to 7 show results of several transcripts differentially expressed in the cardiac arrest model over several time points. Each sequence is examined in their expression levels over a time period of 0.5 hrs after cardiac arrest to 2 years after surgery and compared to sham operated controls. Genes are described as differentially expressed when the sequence is up- or down-regulated at one or more time points with a certain statistical relevant significance value. Over time the expression pattern can be determined as up-regulated in one to seven time points; as down regulated in one to seven time points or as mixed regulated if the type of regulation changes between up- and down-regulation at different time points.

X-axis describes the time points analyzed by DEPD, 0.5 h=0.5 hrs post operation, 1 h+1 hr post operation, 3 h=3 hrs post operation, 6 h=6 hrs post operation, 3 d=day 3 post operation, 7 d=day 7 post operation, 2 y=2 years post operation.

The Y-axis shows delta h, which represents the normalized difference of expression (peak height) of a certain transcript between a control group and a treated group. x-fold difference in gene expression is calculated by:

1+delta h/1−delta h

0=no change to control, +=up regulation, −=down regulation; (0.2=1.5 fold; 0.3=1.86 fold; 0.4=2.33 fold; 0.5=3 fold).

### EXAMPLE 2

### Determination of Expression Levels

Gene expression profiling by DEPD-analysis starts with the isolation of 5-10 μg total RNA. In a second step, double-stranded cDNA is synthesized. Through an enzymatic digest of the cDNA with three different type IIS restriction enzymes, three pools with short DNA-fragments containing single-stranded overhangs are generated. Afterwards, specific DNA-adaptor-molecules are ligated and in two subsequent steps 3.072 PCR reactions are performed by using 1024 different unlabelled 5′ primer and a common FAM-fluorescent-labelled 3′-primer in the last PCR step. Subsequently, the 3072 PCR pools are analyzed on an automatic capillary electrophoresis sequencer.

Differential gene expression pattern of single fragments are determined by comparison of normalized chromatogram peaks from the control groups and corresponding operated animals.

### EXAMPLE 3

### Sequencing and Databank Analysis of the Obtained Sequences

Differentially expressed peaks are confirmed on polyacrylamide gels by using radioactive labelled 3′ primer instead of the FAM fluorescent primer. Differentially expressed bands are cut from the gel. After an elution step of up to 2 hrs in 60 μl 10 mM Tris pH 8, fragments are re-amplified by PCR using the same primer as used in the DEPD analysis. Resulting PCR products are treated with a mixture of Exonuclease I and shrimp alkaline phosphatase prior to direct sequencing. Sequencing reactions are performed by using DYEnamic-ET-dye terminator sequencing kit (Amersham) and subsequently analyzed by capillary electrophoresis (Megabace 1000, Amersham).

Prior to a BLAST sequence analysis (Altschul et al. 1997 Nucleic Acids Res 25:3389-3402) against GenBank (Release No. 126), all sequences are quality verified and redundant sequences or repetitive motifs are masked.

| | | | |
|---|---|---|---|
| SEQ- | accession | | fragment |
| ID | number | name | length [bp] |

| | | | |
|---|---|---|---|
| 1 | Z99755 | Human DNA sequence from clone CTA-714B7 on | 220 |
| | | chromosome 22q12.2-13.2 Contains pseudogene | |
| | | similar to part of COX7B (Cytoclirome c oxidase | |
| | | subunit VIIb) | |
| 2 | | no hit found in DB | 296 |
| 3 | BF418899 | UI-R-BJ2-bqk-c-03-0-UI.s1 UI-R-BJ2 Rattus | 287 |
| | | norvegicus cDNA clone UI-R-BJ2-bqk-c-03-0-UI 3′, | |
| | | mRNA sequence | |
| 4 | | no hit found in DB | 91 |
| 5 | AB023781 | Rattus norvegicus mRNA for cathepsin Y, partial cds | 258 |
| 6 | BF420410 | UI-R-BJ2-bqb-g-04-0-UI.s1 UI-R-BJ2 Rattus | 200 |
| | | norvegicus cDNA clone UI-R-BJ2-bqb-g-04-0-UI 3′, | |
| | | mRNA sequence | |
| 7 | M35826 | Rat mitochondrial NADH-dehydrogenase (NDI) gene, | 347 |
| | | complete cds | |
| 8 | AK019199 | Mus musculus 11 days embryo cDNA, RIKEN full- | 183 |
| | | length enriched library, clone:2700005I17, full insert | |
| | | sequence | |
| 9 | BF413204 | UI-R-BT1-bny-a-04-0-UI.s1 UI-R-BT1 Rattus | 214 |
| | | norvegicus cDNA clone UI-R-BT1-bny-a-04-0-UI 3′, | |
| | | mRNA sequence | |
| 10 | X16555 | Rat PRPS2 mRNA for phosphoribosylpyrophosphate | 159 |
| | | synthetase subunit II | |
| 11 | AK017685 | Mus musculus 8 days embryo cDNA, RIKEN full- | 195 |
| | | length enriched library, clone:5730466L18, full insert | |
| | | sequence | |
| 12 | | no hit found in DB | 164 |
| 13 | Y00964 | M. musculus mRNA for beta-hexosaminidase | 181 |
| 14 | | no hit found in DB | 110 |
| 15 | L17127 | Rattus norvegicus proteasome RN3 subunit mRNA, | 274 |
| | | complete cds | |
| 16 | | no hit found in DB | 132 |
| 17 | BG380139 | UI-R-CS0-btp-e-04-0-UI.s1 UI-R-CS0 Rattus | 139 |
| | | norvegicus cDNA clone UI-R-CS0-btp-e-04-0-UI 3′, | |
| | | mRNA sequence | |
| 18 | D86215 | Rattus norvegicus mRNA for NADH:ubiquinone | 276 |
| | | oxidoreductase, complete cds | |
| 19 | AK005320 | Mus musculus adult male cerebellum cDNA, RIKEN | 129 |
| | | full-length enriched library, clone:1500031J01, full | |
| | | insert sequence | |
| 20 | | no hit found in DB | 118 |
| 21 | | no hit found in DB | 154 |
| 22 | | no hit found in DB | 103 |
| 23 | | no hit found in DB | 73 |
| 24 | | no hit found in DB | 107 |
| 25 | BI287855 | UI-R-CW0s-ccm-a-07-0-UI.s1 UI-R-CW0s Rattus | 217 |
| | | norvegicus cDNA clone UI-R-CW0s-ccm-a-07-0-UI 3′, | |
| | | mRNA sequence | |
| 26 | | no hit found in DB | 151 |
| 27 | | no hit found in DB | 200 |
| 28 | AC016673 | Homo sapiens BAC clone RP11-17N4 from 2, | 217 |
| | | complete sequence | |
| 29 | AB071989 | Mus musculus mRNA for Spop, partial cds | 310 |
| 30 | | no hit found in DB | 211 |
| 31 | AF178845 | Rattus norvegicus calmodulin mRNA, complete cds | 410 |
| 32 | J02701 | Rat Na+, K+-ATPase beta subunit protein mRNA, | 341 |
| | | complete cds | |
| 33 | X12553 | Rat mRNA for liver Cytochrome c oxidase subunit VIa | 411 |
| 34 | | no hit found in DB | 118 |
| 35 | Y07503 | Rat mRNA for preproenkephalin (A) | 186 |
| 36 | X14876 | Rat mRNA for transthyretin | 205 |
| 37 | | no hit found in DB | 202 |
| 38 | AI54730 | UI-R-C3-sr-b-11-0-UI.s1 UI-R-C3 Rattus norvegicus | 200 |
| | | cDNA clone UI-R-C3-sr-b-11-0-UI 3′, mRNA | |
| | | sequence | |
| 39 | M27315 | Rattus norvegicus Cytochrome c oxidase subunit II (Co | 146 |
| | | II) gene | |
| 40 | J02582 | Rat apolipoprotein E gene, complete cds | 139 |
| 41 | | no hit found in DB | 234 |
| 42 | U65579 | Human mitochondrial NADH dehydrogenase- | 291 |
| | | ubiquinone Fe-S protein 8, 23 kDa subunit precursor | |
| | | (NDUFS8) nuclear mRNA encoding mitochondrial | |
| | | protein, complete cds | |
| 43 | AF173082 | Mus musculus LIN-7 homolog 2 (MALS-2) mRNA, | 113 |
| | | complete cds | |
| 44 | | no hit found in DB | 197 |
| 45 | U70268 | Rattus norvegicus mud-7 mRNA, 3′ UTR | 206 |
| 46 | X96997 | O.aries SOX-2 gene | 146 |
| 47 | AK020957 | Mus musculus adult male corpora quadrigemina | 181 |
| | | cDNA, RIKEN full-length enriched library, | |
| | | clone:B230104P22, full insert sequence | |
| 48 | | no hit found in DB | 272 |
| 49 | BC012314 | Mus musculus, Similar to ferritin heavy chain, clone | 73 |
| | | MGC:19422 IMAGE:3488821, mRNA, complete cds | |
| 50 | BB452941 | BB452941 RIKEN full-length enriched, 12 days | 125 |
| | | embryo spinal ganglion Mus musculus cDNA clone | |
| | | D130020J05 3′, mRNA sequence | |
| 51 | AK019418 | Mus musculus 13 days embryo head cDNA, RIKEN | 365 |
| | | full-length enriched library, clone:3110018K01, full | |
| | | insert sequence | |
| 52 | J01435 | Rattus norvegicus mitochondrial ATPase subunit 6 | 175 |
| | | gene | |
| 53 | BF387893 | UI-R-CA1-bbw-a-03-0-UI.s1 UI-R-CA1 Rattus | 182 |
| | | norvegicus cDNA clone UI-R-CA1-bbw-a-03-0-UI 3′, | |
| | | mRNA sequence | |
| 54 | AB033713 | Rattus norvegicus mitochondrial gene for Cytochrome | 101 |
| | | b, partial cds | |
| 55 | | no hit found in DB | 123 |
| 56 | U53513 | Rattus norvegicus glycine-, glutamate-, | 76 |
| | | thienylcyclohexylpiperidine-binding protein mRNA, | |
| | | complete cds | |
| 57 | AK004546 | Mus musculus adult male lung cDNA, RIKEN full- | 261 |
| | | length enriched library, clone:1200002H13, full insert | |
| | | sequence | |
| 58 | | no hit found in DB | 269 |
| 59 | AY004290 | Rattus norvegicus scg10-like-protein mRNA, complete | 277 |
| | | cds | |
| 60 | | no hit found in DB | 228 |
| 61 | | no hit found in DB | 119 |
| 62 | BF544005 | UI-R-E0-ce-c-04-0-UI.r1 UI-R-E0 Rattus norvegicus | 117 |
| | | cDNA clone UI-R-E0-ce-c-04-0-UI 5′, mRNA | |
| | | sequence | |
| 63 | | no hit found in DB | 217 |
| 64 | | no hit found in DB | 269 |
| 65 | | no hit found in DB | 162 |
| 66 | BC011132 | Mus musculus, Similar to special AT-rich sequence | 200 |
| | | binding protein 1, clone MGC:18461 | |
| | | IMAGE:4164993, mRNA, complete cds | |
| 67 | AJ278701 | Rattus norvegicus mRNA for cytosolic branched chain | 182 |
| | | aminotransferase (Bcatc gene) | |
| 68 | M14512 | Rat Na⁺,K⁺-ATPase alpha(+) isoform catalytic subunit | 207 |
| | | mRNA, complete cds | |
| 69 | U42975 | Rattus norvegicus Shal-related potassium channel | 268 |
| | | Kv4.3 mRNA, complete cds | |
| 70 | BC004706 | Mus musculus, heterogeneous nuclear | 285 |
| | | ribonucleoprotein C, clone MGC:5715 | |
| | | IMAGE:3499283, mRNA, complete cds | |
| 71 | BE101398 | UI-R-BJ1-aud-e-10-0-UI.s1 UI-R-BJ1 Rattus | 158 |
| | | norvegicus cDNA clone UI-R-BJ1-aud-e-10-0-UI 3′, | |
| | | mRNA sequence | |
| 72 | AF073297 | Mus musculus small EDRK-rich factor 2 (Serf2) | 144 |
| | | mRNA, complete cds | |
| 73 | D32249 | Rattus norvegicus mRNA for neurodegeneration | 250 |
| | | associated protein 1, complete cds | |
| 74 | BF391228 | UI-R-CA1-bcq-g-07-0-UI.s1 UI-R-CA1 Rattus | 122 |
| | | norvegicus cDNA clone UI-R-CA1-bcq-g-07-0-UI 3′, | |
| | | mRNA sequence | |
| 75 | BE109851 | UI-R-CA0-axi-b-04-0-UI.s1 UI-R-CA0 Rattus | 157 |
| | | norvegicus cDNA clone UI-R-CA0-axi-b-04-0-UI 3′, | |
| | | mRNA sequence | |
| 76 | | no hit found in DB | 107 |
| 77 | AY011335 | Rattus norvegicus amyloid beta precursor protein | 254 |
| | | (App) gene, partial cds | |
| 78 | BC013540 | Mus musculus, Similar to retinal short-chain | 208 |
| | | dehydrogenase/reductase 1, clone MGC:19224 | |
| | | IMAGE:4241608, mRNA, complete cds | |
| 79 | X52311 | Rat unr mRNA for unr protein with unknown function | 300 |
| 80 | M29358 | Rat ribosomal protein S6 mRNA, complete cds | 192 |
| 81 | AC068987 | UI-R-CA0-bkh-f-06-0-UI.s1 UI-R-CA0 Rattus | 300 |
| | | norvegicus cDNA clone UI-R-CA0-bkh-f-06-0-UI 3′, | |
| | | mRNA sequence | |
| 82 | AC068987 | UI-R-BO1-aqb-b-02-0-UI.s1 UI-R-BO1 Rattus | 328 |
| | | norvegicus cDNA clone UI-R-BO1-aqb-b-02-0-UI 3′, | |
| | | mRNA sequence | |
| 83 | | no hit found in DB | 186 |
| 84 | M23953 | UI-R-BJ0p-aio-h-09-0-UI.s1 UI-R-BJ0p Rattus | 370 |
| | | norvegicus cDNA clone UI-R-BJ0p-aio-h-09-0-UI 3′, | |
| | | mRNA sequence | |
| 85 | AK018721 | Mus musculus adult male kidney cDNA, RIKEN full- | 441 |
| | | length enriched library, clone:0610007M20, full insert | |
| | | sequence | |
| 86 | X82396 | R. norvegicus mRNA for cathepsin B | 412 |
| 87 | | no hit found in DB | 149 |
| 88 | X82550 | R. norvegicus mRNA for ribosomal protein L41 | 207 |

DB = data base

### EXAMPLE 4

### Comparison of Differentially Expressed Sequences Over Several Time Points in the Cardiac Arrest Model

0.5 hr, 1 hr, 3 hrs, 6 hrs, 3 days, 7 days, and 2 years survival time of the animals were chosen as time points for gene expression profiling of the cardiac arrest model. After DEPD analysis peaks obtained as differentially expressed at least at one time point were compared over time to control within the cardiac arrest stroke model. Results are shown in Table 2.

TABLE 2

| | |
|---|---|
| SEQ-ID | |
| No. | Regulation |

| | |
|---|---|
| 1 | down 7d |
| 2 | down 7d |
| 3 | down 7d |
| 4 | down 7d |
| 5 | up 3d/up 7d |
| 6 | up 3d |
| 7 | up 3d |
| 8 | down 3d |
| 9 | down 3d |
| 10 | up 3d |
| 11 | down 3d |
| 12 | up 3d |
| 13 | up 3d |
| 14 | up 7d |
| 15 | up 7d |
| 16 | up 3d |
| 17 | down 7d |
| 18 | up 7d |
| 19 | down 7d |
| 20 | down 7d |
| 21 | up 7d |
| 22 | up 3d/down 7d |
| 23 | up 3d |
| 24 | down 3d |
| 25 | down 7d |
| 26 | up 3d |
| 27 | down 3d |
| 28 | up 3d |
| 29 | down 3d |
| 30 | down 3d |
| 31 | down 7d |
| 32 | up 3d |
| 33 | up 7d |
| 34 | up 3d |
| 35 | up 3d/up 7d |
| 36 | up 7d |
| 37 | up 7d |
| 38 | down 7d |
| 39 | up 7d |
| 40 | up 7d |
| 41 | down 7d |
| 42 | up 7d |
| 43 | up 7d |
| 44 | down 7d |
| 45 | down 7d |
| 46 | up 7d |
| 47 | up 7d |
| 48 | down 7d |
| 49 | up 3d |
| 50 | down 7d |
| 51 | down 7d |
| 52 | up 7d |
| 53 | up 3d |
| 54 | up 3d |
| 55 | up 3d |
| 56 | up 3d |
| 57 | down 3d |
| 58 | down 3d |
| 59 | down 3d |
| 60 | down 3d |
| 61 | up 3d |
| 62 | down 3d |
| 63 | up 3d |
| 64 | up 3d |
| 65 | up 3d |
| 66 | up 3d/down 7d |
| 67 | up 3d |
| 68 | up 3d |
| 69 | down 3d |
| 70 | down 3d/down |
| | 7d |
| 71 | up 3d |
| 72 | up 3d |
| 73 | up 3d |
| 74 | down 3d |
| 75 | down 2y |
| 76 | up 2y |
| 77 | up 2y |
| 78 | down 2y |
| 79 | down 2y |
| 80 | down 2y |
| 81 | up 2y |
| 82 | up 2y |
| 83 | up 2y |
| 84 | up 2y |
| 85 | up 2y |
| 86 | down 2y |
| 87 | up 2y |
| 88 | up 2y |

For each DNA fragment, gene expression patterns are obtained in the stroke model compared over several time points. “Up”, “down”, and “mixed” is defined as time dependent expression at one or more time points in the cardiac arrest model compared to the non-disease model.

What is claimed is:

1. A method for characterizing a stroke status, the development and/or occurrence of stroke, and/or the progression of the pathology of stroke, and/or consequences of stroke, comprising:
a) detecting a level of expression of at least two gene sequences selected from the group consisting of SEQ ID NO. 1 to 88 or homologues or fragments thereof, in a test sample from an individual with stroke;
b) detecting a level of expression of said at least two gene sequences in a control sample; and
c) comparing the levels of expression in the test and control samples, thereby characterizing the stroke status.

2. The method of claim 1, wherein the control sample is from an individual without stroke.

3. The method of claim 1, wherein the control sample is derived from a cell line.

4. The method of claim 1, wherein said individual is an animal.

5. The method of claim 1, wherein said individual is a human.

6. The method of claim 2, wherein said individual is an animal.

7. The method of claim 2, wherein said individual is a human.

8. The method of claim 1, wherein the test sample and control sample are derived independently from a source selected from the group consisting of whole tissues, cerebrospinal fluid, blood, isolated cells, and cell lines.

9. The method of claim 1, wherein the test sample and control sample are derived independently from an in vivo sample, an in vitro sample, or an ex vivo sample.

10. The method of claim 1, wherein the levels of expression in the test sample are increased relative to the levels of expression in the control sample.

11. The method of claim 1, wherein the levels of expression in the test sample are decreased relative to the levels of expression in the control sample.

12. The method of claim 1, further comprising detecting a level of expression of said at least two gene sequences in the test sample prior to and following administration of a stroke treatment; and comparing the levels of expression prior to and following administration, thereby assessing the efficacy of the stroke treatment.

13. The method of claim 1, wherein said individual is an animal used in an animal model for studying stroke.

14. The method of claim 13, wherein the animal is subjected to a stroke.

15. The method of claim 14, wherein the animal is administered a compound that may alter the stroke status.

16. The method of claim 1, further comprising detecting at least four gene sequences selected from the group consisting of SEQ ID NO. 1 to 88 or homologues or fragments thereof, in the test sample and the control sample.

17. The method of claim 1, further comprising detecting at least six gene sequences selected from the group consisting of SEQ ID NO. 1 to 88 or homologues or fragments thereof, in the test sample and the control sample.

18. The method of claim 1, further comprising detecting at least eight gene sequences selected from the group consisting of SEQ ID NO. 1 to 88 or homologues or fragments thereof, in the test sample and the control sample.

19. The method of claim 1, further comprising detecting at least ten gene sequences selected from the group consisting of SEQ ID NO. 1 to 88 or homologues or fragments thereof, in the test sample and the control sample.

20. The method of claim 1, wherein said stroke is ischemic stroke.

21. The method of claim 1, wherein said stroke is hemorrhagic stroke.

22. The method of claim 1, wherein detecting the level of expression in the test sample and control sample further comprises at least one method selected from the group consisting of PCR of a cDNA, hybridization of a sample DNA, and detecting one or more polypeptides encoded by said at least two gene sequences or homologues or fragments thereof.

23. A method for characterizing a stroke status, the development and/or occurrence of stroke, and/or the progression of the pathology of stroke, and/or consequences of stroke, comprising:
a) providing a test sample comprising a cell or a body fluid expressing a polynucleotide sequence selected from the group consisting of SEQ ID NO. 1 to 88 or homologues or fragments thereof;
b) detecting expression of said polynucleotide in said test sample;
c) comparing the expression of said polynucleotide in said test sample to expression of the same polynucleotide in a reference sample whose expression stage is known; and
d) identifying a difference in the levels of expression between said test sample and said reference sample, thereby characterizing the stroke status.

24. A method for identifying a therapeutic agent for treating stroke in a subject, comprising:
a) providing a test cell capable of expressing a polynucleotide sequence selected from the group consisting of SEQ ID NO: 1 to 88 or homologues or fragments thereof;
b) detecting expression of said polynucleotide sequence in said test cell;
c) contacting said test cell with the therapeutic agent;
d) detecting expression of said polynucleotide sequence in said test cell contacted with the therapeutic agent;
e) comparing the expression of said polynucleotide sequence in step (b) to the expression of said polynucleotide sequence in step (d); and
f) identifying a change in expression of said polynucleotide in the presence of the therapeutic agent, thereby identifying the therapeutic agent for treating stroke.

25. The method of claim 24, wherein said stroke is ischemic.

26. The method of claim 24, wherein said stroke is hemorrhagic.

27. The method of claim 24, wherein detecting expression of said polynucleotide further comprises at least one method selected from the group consisting of PCR of a cDNA, hybridization of a sample DNA, and detecting a polypeptide encoded by said polynucleotide or homologue or fragment thereof.

28. A pharmaceutical composition, comprising a polynucleotide selected from the group consisting of SEQ ID NO: 1 to 88 or homologues or fragments thereof, or a polypeptide encoded by said polynucleotide.

29. A kit comprising a reagent for detecting a polynucleotide selected from the group consisting of SEQ ID NO: 1 to 88 or homologues or fragments thereof.

30. A vector, comprising a polynucleotide selected from the group consisting of SEQ ID NO: 1 to 88 or homologues or fragments thereof.

31. A host cell, comprising a polynucleotide selected from the group consisting of SEQ ID NO: 1 to 88 or homologues or fragments thereof.

32. An antibody that selectively binds to a polypeptide encoded by a polynucleotide selected from the group consisting of SEQ ID NO: 1 to 88 or homologues or fragments thereof.

33. A transgenic animal, comprising a polynucleotide selected from the group consisting of SEQ ID NO: 1 to 88 or homologues or fragments thereof, wherein said polynucleotide has been altered compared to a wild type phenotype.
